SEQUENCE LISTING (5 sequences)

SEQ ID NO: 1 - DNA, 717 bp, Artificial
  synthetic plasmid insert
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcctgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 2 - DNA, 717 bp, Artificial
  synthetic plasmid insert
atgagcaaaggagaagaacttttcactggagttgttccaattcttgttgaattagatggtgatgttaacggtcacaaattttctgtcagtggagagggtgaaggtgatgcaacatacggaaaacttacccttaaatttatttgcaccacggggaagctacccgtcccctggcccacccttgtcaccacgttcggttatggtgttcaatgctttgcgagatacccagatcatatgaaacagcatgactttttcaagagtgccatgcctgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactctcacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgcgctttcgaaagatcccaacgaaaagagagaccacatggtccttcttgagtttgtaacagctgctgggattacacatggcatggatgaactatacaaataa

SEQ ID NO: 3 - DNA, 717 bp, Artificial
  synthetic plasmid insert
atgagtaaaggagaagaacttttcactggagtcgtccccattctggttgagttagatggtgatgttaacggtcacaaattctctgttagcggtgaaggtgaaggtgatgcaacatacggaaaacttactcttaaatttatttgcactactggtaaacttcctgttccatggccaacacttgtcactactttcggttatggtgttcaatgctttgcgagatacccagatcatatgaaacagcatgactttttcaagagtgccatgcctgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 4 - DNA, 717 bp, Artificial
  synthetic plasmid insert
atgagtaaaggagaagaactgttcactggagttgtcccaattctggttgaactggatggtgatgttaatggtcacaaattttctgtcagtggagagggtgaaggtgatgcaacatacggaaaacttacccttaaatttatttgcactactgggaaactacccgtaccgtggcccacgctagtcactactttcgggtatggggtacaatgctttgcgaggtacccagatcatatgaaacagcatgactttttcaagagtgccatgcctgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 5 - DNA, 717 bp, Artificial
  synthetic plasmid insert
atgagtaaaggagaagaacttttcactggggttgtcccgattctagtagaattagatggggatgtaaatgggcacaaattttctgtcagtggagagggtgaaggtgatgctacatacggaaaactgaccctgaaatttatttgcactactggtaaactgcctgttccatggccaacactggtcactactttcggttatggtgttcaatgctttgcgagatacccagatcatatgaaacagcatgactttttcaagagtgccatgcctgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

(19) United States
(12) Patent Application Publication
(10) Pub. No.: US 2013/0203113 A1
(43) Pub. Date: Aug. 8, 2013

(54) METHOD OF STABILIZING mRNA

(71) Applicants: Steen Pedersen, Birkeroed (DK); Kim Sneppen, Charlottenlund (DK); Margit Pedersen, Roskilde (DK); Sine Lo Svenningsen, Koebenhavn K (DK); Namiko Mitarai, Koebenhavn N (DK)

(72) Inventors: Steen Pedersen, Birkeroed (DK); Kim Sneppen, Charlottenlund (DK); Margit Pedersen, Roskilde (DK); Sine Lo Svenningsen, Koebenhavn K (DK); Namiko Mitarai, Koebenhavn N (DK)

(73) Assignee: Kobenhavns Universitet, Copenhagen N (DK)

(21) Appl. No.: 13/696,885

(22) PCT Filed: May 5, 2011
(86) PCT No.: PCT/DK2011/050153

§ 371 Date: Jan. 24, 2013

Related U.S. Application Data
(60) Provisional application No. 61/332,749, filed on May 8, 2010.

(30) Foreign Application Priority Data
May 8, 2010 (DK) PA 2010 70194

(51) Int. Cl. C12N 15/67; C12P 21/00

(52) U.S. Cl. CPC C12N 15/67; C12P 21/00
USPC 435/69.1; 435/320.1; 435/252.31; 435/252.33; 435/254.21; 435/254.23; 435/254.3; 435/419; 435/414; 435/358; 435/367; 435/346

(57) ABSTRACT

There is provided a method to increase the production of a desired protein in a microorganism by introduction of slowly translated codons in the encoding DNA gene sequence capable of slowing down the translation speed of the ribosomes moving along the mRNA, whereby the ribosomes protect the mRNA from being enzymatically degraded. This increases the stability of the mRNA transcript and thus results in an increased production of the desired protein. Moreover, there is provided a method of decreasing the half-life of a mRNA transcript from a gene encoding a peptide.

### FIELD OF THE INVENTION

The present invention relates to a method for increasing the production of a desired protein in bacteria, fungi, plant and animal cells. More specifically this is achieved by introduction of slowly translated codons in the encoding DNA gene sequence. Moreover, there is provided a method of decreasing the half-life of a mRNA transcript from a gene encoding a peptide.

### BACKGROUND OF THE INVENTION

Increasing the levels of transcription of a gene is well known in the art to lead to higher levels of the protein encoded by the overexpressed gene. It is also well known in the art that overproduction of proteins by means of transcription overexpression may lead to undesirable effects on cellular metabolism (WO 98/07846). Furthermore, it has also been described that protein overproduction may lead to deleterious effects in the translational machinery of the host cell (Hengjiang et al., 1995, J. Bacteriol. 177.1497-1504) and/or induction of proteolytic activities mediated by stress responses (Ramirez D. M., and W. E. Bentley, 1995, Biotechnol. Bioeng. 47:596-608) which could be the consequence of lower production titers.

Therefore, devising methods for protein overproduction alternative to the use of constitutive strong promoters could be advantageous.

Transcript degradation is utilized by microorganisms as a means to control cellular protein content. On the other hand, microorganisms have developed mechanisms by which the stability of a given transcript is enhanced. To achieve this, transcripts are provided with nucleotide sequences capable of forming secondary structures which impose an impediment for mRNA degrading enzymes to exert their action.

Smolke et al. (2001, Metabolic Engineering. 3: 313-321) describe the use of artificially generated sequences capable of stem-loop structure formation as mRNA stability elements to increase the steady-state level of transcripts encoded by two plasmid-borne crt genes in order to increase phytoene production in Escherichia coli. For this method to be useful, the above-mentioned mRNA stability elements must be precisely placed no more than one nucleotide away from a promoter transcriptional start site (Carrier and Keasling 1999, Biotechnol. Prob. 1, 5: 58-64). Alternatively, if cleavage is desired at a site within the native mRNA molecules, the mRNA stabilizing element is required to be co-introduced with an RNase E cleavage site so that RNase E—specific cleavage results in a new mRNA molecule of similar structure, i.e. placement of the RNA stability element one (1) nucleotide from the 5′ end. Either example requires laborious experimental work, limiting the usefulness of the method.

Thus the development of stabilizing mRNA independent of promoters at the transcriptional start sites or independent of RNase E cleavage could offer a better alternative to engineer microorganisms for the manufacture of proteins at the industrial level.

Most mRNA in E. coli decay with functional half-lives close to two minutes at 37° C., but a few mRNA species differ substantially in their half-life resulting in a span among mRNA half-lives of close to 100-fold (Blundell et al, 1972, Pedersen et al, 1978, Gerdes et al, 1990). The extraordinary stability of the latter mRNA depends on sequestering of the mRNAs 5′ end into a structure (Franch et al, 1997). Characterization of mutants with altered mRNA half-lives has led to models for the mRNA degradation where endonuclease RNaseE, the exonucleases RNase II and RNase R, polynucleotide phosphorylase (Babitzke and Kushner 1981, Donovan and Kushner, 1986, Cheng and Deutcher, 2005) and polyA-polymerase I that poly-adenylates the 3′ end of the mRNA combine to form a complex, a “degradosome” responsible for the decay of the mRNA (Yarchuk et al 1992, Dreyfus and Regnier, 2002; Kushner, 2002; Deana and Belasco, 2005). It is likely that separate pathways for the degradation of specific mRNAs exist (Deana and Belasco, 2005; Carabetta et al 2009).

Attempts have been made to characterize the initial event that specifies the inactivation of an mRNA that is followed by a rapid chemical degradation of the mRNA. Petersen (1987) constructed eight variants of the lacZ mRNA with small sequences inserted in the early coding part of the mRNA and determined their functional half-lives and translation initiation frequencies. These changes decreased the mRNA half-life but the half-life did not appear to be influenced by the translation initiation frequency or by hairpin mRNA structures early in the coding region. By contrast, by introducing wild type and mutated ribosome binding sites from other genes into the lacZ gene, Yarchuk et al, (1992) got results indicating that cleavage by RNaseE was the rate limiting step for mRNA degradation and that the rate of such cleavage was influenced by the translation initiation frequency. When the lacZ ribosome-binding site was substituted with sites expected to bind ribosomes with a higher affinity, the levels of protein expression from these constructs were increased. This was largely due to an increased mRNA half-life and only marginally due to an increased rate of translation initiation (Vind et al, 1993). This indicated that small increases in the ribosome density on an mRNA increased its half-life substantially. In general, the translation efficiency of the mRNA has a large influence on its stability but the event that initiates the decay and determines the functional half-life of the mRNA was therefore elusive (reviewed in Deana and Belasco, 2005).

Recently, the hydrolysis of the 5′ tri-phosphate to a 5′ mono-phosphate group at the end of the mRNA, catalysed by the RppH enzyme, was suggested to be an initial and rate-limiting step in the mRNA degradation (Celesnik et al 2007, Deana et al 2008). Secondary mRNA structures in the 5′ untranslated region were shown to protect the 5′ tri-phosphate group and to stabilize the mRNA. However, the mRNAs characterized by Petersen (1987) were identical with respect to the initial 52 nucleotides of the lacZ mRNA which includes the first fifteen nucleotides of the coding region and thus did not vary in the 5′-untranslated region. Nevertheless, minor sequence changes shortly after codon 5 resulted in an up to four-fold decrease of the mRNA half-life.

Recently the translation process was modelled with focus on kinetic data where translation of lacZ mRNA with inserts of slowly translated codons indicated the formation of ribosome queues. This allowed estimation of translation initiation rate on lacZ mRNA in living E. coli rather precisely to 1 initiation per 2.3 sec under the conditions used, growth in glycerol minimal medium. This analysis also indicated that stochastic collisions between ribosomes are normal, frequent and probably harmless events (Mitarai et al 2008). Because it takes approximately one second to translate the 11 codons that is covered by a ribosome, the distance between the ribosomes translating the lacZ mRNA is on average just above one ribosome diameter, subject to varying local translation rates and to stochastic fluctuations. The translation rate among individual codons varies approximately ten-fold (Sørensen and Pedersen 1991), enough to give large local variations in the spacing of the ribosomes even with an identical translation initiation frequency.

Consequently, it is an object of the present invention to provide a method to increase the production of a desired protein in a microorganism without strengthening native promoter signals controlling transcription of said structural gene sequences.

### SUMMARY OF THE INVENTION

The inventors of the present invention have used a refined modelling to be able to analyse the ribosome distribution on different mRNA sequences in quantitative terms. Using this refined model on lacZ variant mRNAs with either altered ribosome-binding sites, or with changed codons in the early coding part of the mRNA, the inventors surprisingly found a clear correlation between the mRNAs functional half-life and the fraction of time an initial part of the mRNA is uncovered by ribosomes. These findings have been verified with in vivo.

Based on these findings the present inventors have contemplated a method to increase the production of a desired protein in a microorganism by introduction of one or more slowly translated codons in the encoding DNA gene sequence capable of slowing down the translation speed of the ribosomes moving along the mRNA, whereby the ribosomes protect the mRNA from being enzymatically degraded. This increases the stability of the mRNA transcript and thus results in an increased production of the desired protein.

In a first aspect the present invention provides a method to increase the production of a desired peptide in a cell by increasing the half-life of the mRNA transcript from the gene encoding the peptide, said method characterized in that one or more slowly translated codons are introduced in the gene 45 or more codons down-stream of the start site of the open reading frame, wherein the one or more slowly translated codons are selected so that the encoded amino acid sequence of the peptide is unchanged as compared to the wild type peptide.

In a preferred embodiment of the present invention the one or more slowly translated codons are introduced in the gene at 45-90, preferably 45-88, more preferably 45-72, and most preferably 45-66, codons down-stream of the start site of the open reading frame.

Preferably the one or more slowly translated codons are selected from codons that are translated with a rate of less than 6 codons per sec.

A preferred cell is a microorganism selected from the group consisting of bacteria, fungi and algae. In a particularly preferred embodiment the microorganism is E. coli. Concerning the gene to be translated the preferred gene is lacZ gene.

Another preferred microorganism is a Bacillus e.g. B. Subtilis, B. megaterium, B. thuringiensis. Still another preferred microorganism is a fungal cell e.g. Saccharomyces cerevisiae, Pichia pastoris, Pichia methanolica, Aspergillus Niger, Aspergillus japonicus

In another embodiment of the present invention the cell is a plant cell e.g. Arabidopsis species, Tobacco species, Medicago species. Alternatively the cells are mammalian cells, e.g. Chinese hamster ovary cells, HeLa cells, hybridoma cells.

In a second aspect the present invention provides a method of increasing the half-life of a mRNA transcript from a gene encoding a peptide, said method characterized in that one or more slowly translated codons are introduced in the gene 45 or more codons down-stream of the start site of the open reading frame, wherein the one or more slowly translated codons are selected so that the encoded amino acid sequence of the peptide is unchanged as compared to the wild type peptide. Preferably, the one or more slowly translated codons are introduced in the gene 45-90 preferably 45-88, more preferably 45-72, and most preferably 45-66, codons down-stream of the start site of the open reading frame. In a very preferred embodiment of the present invention the one or more slowly translated codons are selected from codons that are translated with a rate of less than 6 codons per sec.

In a third aspect the present invention provides a method of decreasing the half-life of a mRNA transcript from a gene encoding a peptide, said method characterized in that one or more slowly translated codons are introduced in the gene 20 or less codons down-stream of the start site of the open reading frame, wherein the one or more slowly translated codons are selected so that the encoded amino acid sequence of the peptide is unchanged as compared to the wild type peptide. Preferably, the one or more slowly translated codons are introduced in the gene 1-20, preferably 4-18, more preferably 5-15, and most preferably 6-15, codons down-stream of the start site of the open reading frame.

Additionally the present invention provides a recombinant vector for increasing the production of a desired peptide in a cell, said vector comprising a DNA sequence encoding the peptide, wherein the DNA sequence has an open reading frame with one or more slowly translated codons introduced 45-72 codons down-stream of the start site of the open reading frame, said one or more slowly translated codons being selected so that the encoded amino acid sequence of the peptide is unchanged as compared to the wild type peptide.

Further the present invention provides a recombinant vector for decreasing the half-life of a mRNA transcribed from the vector encoding a peptide, said vector comprising a DNA sequence with an open reading frame having one or more slowly translated codons introduced 20 or less codons down-stream of the start site of the open reading frame, wherein the one or more slowly translated codons are selected so that the encoded amino acid sequence of the peptide is unchanged as compared to the wild type peptide.

There is also provided host cells transformed with the vectors of the present invention.

Hence the concept of the present invention is to alter codons either before codon 20 or immediately after codon 45 in such a way that codons 20-45 of the mRNA region become either more or less covered with ribosomes. This will stabilize or destabilize the mRNA. To stabilize the mRNA the codon changes should make the codons immediately after codon 45 slower translated compared to the wild type reference; to further stabilize the mRNA the codons before codon 20 may be faster translated. To destabilize the mRNA the codon changes should make the codons before codon 20 slower translated; to further destabilize the mRNA the codons after codon 45 may be faster translated. Also, the codons in the region 20-45 may be changed to faster codons in the case where a mRNA should be destabilized to remove possible ribosome queues in this region.

Concerning the quickly translated codons these are herewith defined as codons that are translated with a rate of more than 8 codons per sec.

The present method of decreasing the half-life of a mRNA transcript may be useful in a number of situations where the reduction of protein titre is of paramount importance:
1) As an alternative to anti-sense mRNA and/or gene knock outs—in case of metabolically important proteins which are essential for the health/operation of the cell but eventually suppress the metabolic pathway.
2) As a method of gene therapy whereby key genes which are over-expressing and which are difficult/impossible to down-regulate are compensated for by replacement with genes engineered to have mRNA with a much lower half-life.
3) By combining a destabilised gene which produces mRNA with poor stability to give a “trickle” of protein with a second copy of the gene which is inducible and produces highly stable mRNA to give high yield of product.

### DESCRIPTION OF THE DRAWINGS

FIG. 1 shows modelling of the ribosome occupancy when translating the first 200 codons in variants of the lacZ mRNA. Panels (a to d) show the fraction of the time each codon is covered by a ribosome for the lacZ variant in: (a) pIV18; (b) pIV1; (c) pCNP1 and (d) pCNP 6 having functional half-lives of 380 sec, 240 sec, 117 sec and 28 sec, respectively. The location of slowly, medium and rapidly translated codons are indicated. The corresponding right panels give the percent of the time a window of 5 codons is free at varying positions of the gene.

FIG. 2 shows correlation between mRNA half-life and the ribosome occupancy of an early part of the mRNA. The mRNA half-life is plotted as a function of the fraction of the time the mRNA from codon 27 to 31 is uncovered by ribosomes. Filled circles, values for the ten lacZ variants used to find the correlation. The values for ribosome occupancy and half-life for the new constructs with either slowly translated codons (one or two of the codons AGG, CGG, GGA see Supplementary Material FIG. S2 for the sequence) at codon 16 or at codon 42; 42, 43, 44 and 42, 43, 44, 45, 46 with half-lives of 26, 116, 120 and 136 respectively see FIG. 3, are indicated on the figure as open circles. The half-life of the reference variant pMAP217 and of pMAP*** with the Shine-Dalgarno sequence from tufA in pIV1 and 5 slow codons at codon 42, 43, 44, 45, 46 is also indicated (open circles).

FIG. 3 shows determination of the functional half-lives of the new lacZ variants constructed to test the model.

FIG. 4 shows unoccupied codons in a window of 5 codons along the first 100 codons in the lacZ wild type (green, t_{0.5}=113* sec) or in variants with slowly translated codons at codon 16, 17 and 18 (red, t_{0.5}=26 sec) or at codon 42 (blue, t_{0.5}=116 sec); at codon 42, 43, 44 (violet, t_{0.5}=120 sec) or at codon 42, 43, 44, 45, 46 (turquoise, t_{0.5}=136 sec). The two vertical lines indicate the mRNA segment from codon 20 to 50.

FIG. 5 shows the modelled ribosome occupancies for the ompA mRNA (top) and the bla mRNA (bottom) plotted as in FIG. 1.

FIG. 6 shows a fraction of total protein that is LacZ protein, plotted as a function of the mRNA half-life (in seconds). In the experiment 35S methionine was incorporated in the growing strains, induced for lacZ expression, and samples were taken after 15, 30 and 45 min. These samples were analyzed on a normal 7.5% SDS-PAGE gel and the amount of LacZ protein and of two proteins, rpoBC that constitutes about 1% of total protein was determined by scanning a PhosphoImager picture of the gel. In the figure the ordinate is the LacZ/rpoBC ratio.

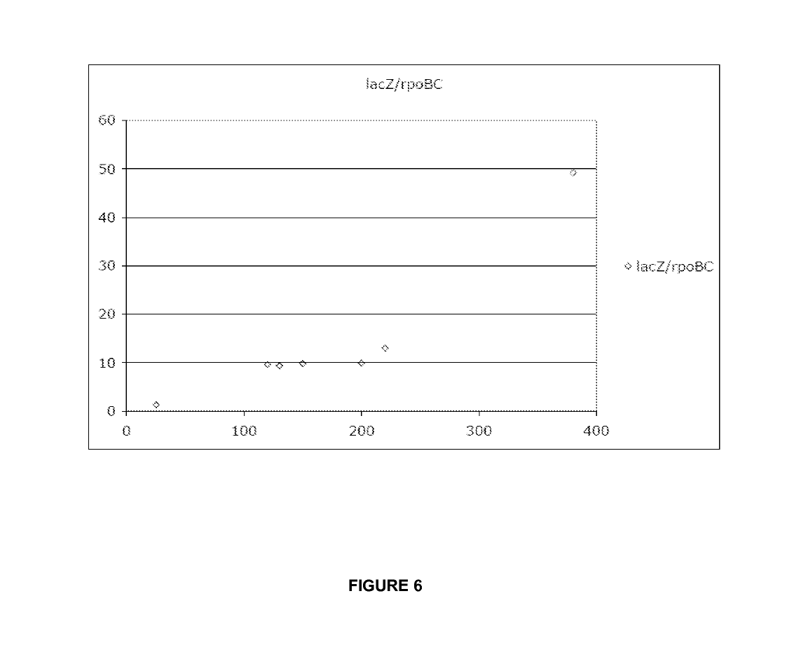

FIG. 7 shows the results from an experiment with CHO cells. pcDNA4/TO containing either wild type GFP construct, stabilized GFP construct, or destabilized construct were used without pcDNA6/TR. This leads to constitutive expression from transfection, and until the plasmids are lost from culture. The results are averages form two measurements from the same culture.

FIG. 8 shows CHO cell cultures transfected with pcDNA6/TR and pcDNA4/TO containing either wild type GFP, stabilized GFP, or destabilized GFP construct. Expression was induced by tetracycline addition for 24 h. (just after “day 1 samples” were taken). Two cultures are made for each construct (wt1 and wt2 are two individual cultures etc.).

FIG. 9 is based on the same data as FIG. 8, averages from (the two) cultures for each construct is used. In this chart is also included a (single) negative control (pcDNA4/TO).

FIG. 10 shows a growth curve for an induction experiment with B. subtilis.

FIG. 11 shows a growth curve for a second induction experiment with B. subtilis.

FIG. 12 shows protein lysates were analyzed by SDS-PAGE in order to visualize the expression of eGFP.

FIG. 13 shows expression of eGFP in the different expression constructs.

FIG. 14 shows qPCR analysis of eGFP mRNA levels in B. subtilis.

### DETAILED DESCRIPTION OF THE INVENTION

Variations in the translation rate of individual codons along an mRNA may cause ribosomes to collide, for instance if slowly translated codons are preceded by rapidly translated codons. The probability of collisions is expected to rise dramatically with the translation initiation frequency. Changes in either the Shine-Dalgarno sequence or in the mRNA coding sequence might therefore affect ribosome spacing quite far from the sequence change itself. To model the distribution of ribosomes along the mRNA in detail the inventors have included additional features to our previous model and applet (Mitarai et al, 2008), which allow for an analysis of the fraction of time a codon is occupied by a ribosome and the fraction of time a specified stretch of mRNA is not masked by ribosomes and therefore possibly accessible for nucleases. The codon specific translation rates used in this modelling were fast (A), middle (B) and slow rate codons (C), translated with a rate of 35; 8; and 4.5 codons per sec, respectively. These values reproduce all our previous determinations of the translation rate in living cells and are therefore a good approximation to the rates used by E. coli (Mitarai et al, 2008).

The inventors first analyse how varying local translation rates will affect the ribosome spacing (FIG. S1 in the Supplementary Materials section). As expected, an even distribution of the fast, average and slowly translated codons leads to an even ribosome spacing; rapidly translated codons located before a stretch of slowly translated codons will be almost totally covered by ribosomes whereas fast codons after a stretch of slowly translated codons will be covered by only few ribosomes. In the three extreme examples given in FIG. S1 the fast-translated codons are covered with ribosomes in 43%, 98% or 8% of the time, respectively.

To analyse more natural mRNAs the inventors turned to the 8 variants of the lacZ mRNA described by Petersen (1987). Here, short sequences inserted between codon 5 and 10 in the lacZ mRNA were found to decrease the mRNA half-life two- to four fold. Also, the inventors analyse translation of lacZ in the two plasmids pIV18 and pIV1 where the lacZ ribosome-binding site was substituted with sequences from highly expressed genes expected to give a stronger ribosome binding compared to lacZ (Vind et al 1993). To be able to model the ribosome spacing on these two mRNA variants the inventors estimated the AG values for the interaction between the Shine-Dalgarno sequences in pIV18 and pIV1 and the 3′ end of 16S ribosomal RNA as described by Freier et al 1986. The interaction affects the off-rate and therefore the resulting on-rate by being proportional to e^{ΔG/RT}. Using this formula, the relative resulting on-rates can be estimated to 1: 18*: 21* for lacZ wild type, tufA and the −9G mutant rpsA mRNA that resulted in a two-respectively three-fold increase in the mRNA half-life for the two latter variants (Vind et al 1993). All together the inventors therefore model data from a total of ten variants in the early lacZ mRNA sequence that experimentally has been shown to give a more than ten-fold change in the functional mRNA half-life. These lacZ variants are all carried on pMLB1034 (Shultz et al 1982) as are the plasmids used by Sørensen and Pedersen (1991) that provided the data that Mitarai et al (2008) modelled to determine the precise rate of initiation for translating the lacZ mRNA 1 initiation per 2.3 sec. Furthermore, all determinations of the functional half-lives were done under the same conditions (same background strain, temperature and growth medium) and the residual syntheses of β-galactosidase were followed after removal of the inducer by filtration and thus without using rifampicin to block the general transcription.

Modelling with these parameters show that the resulting initiation rates for lacZ mRNA translation in the plasmids pIV18 and pIV1 should be only marginally increased, by 14* or 4*% respectively, relative to the lacZ wild type initiation rate, in good agreement with the experimentally determined values (Vind et al 1993). This is due to the time it takes to translate the first eleven codons that constitutes a ribosome diameter. The presence of a ribosome here prevents binding of the following ribosome and prevents the binding-site to be used to its full capacity.

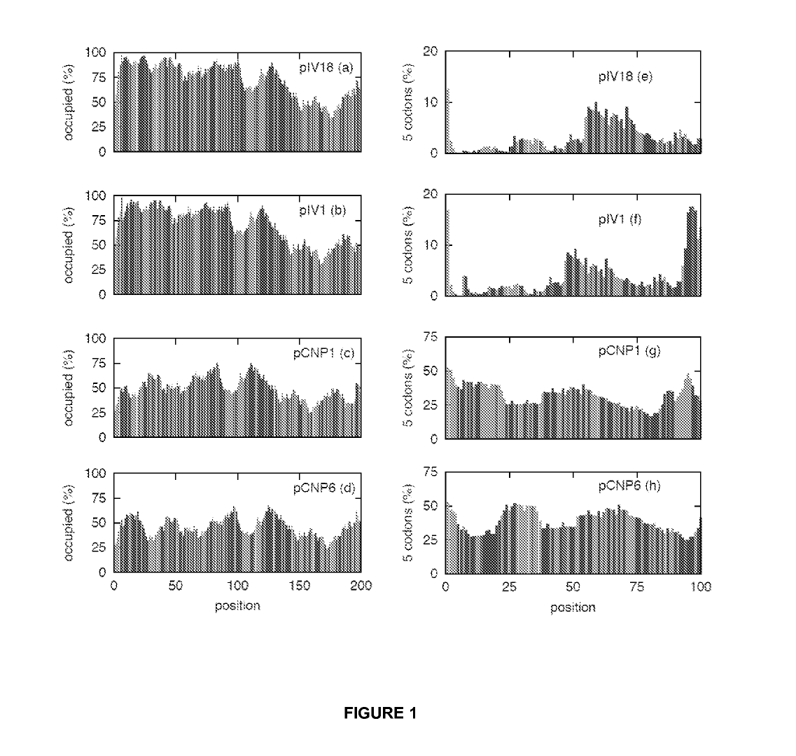

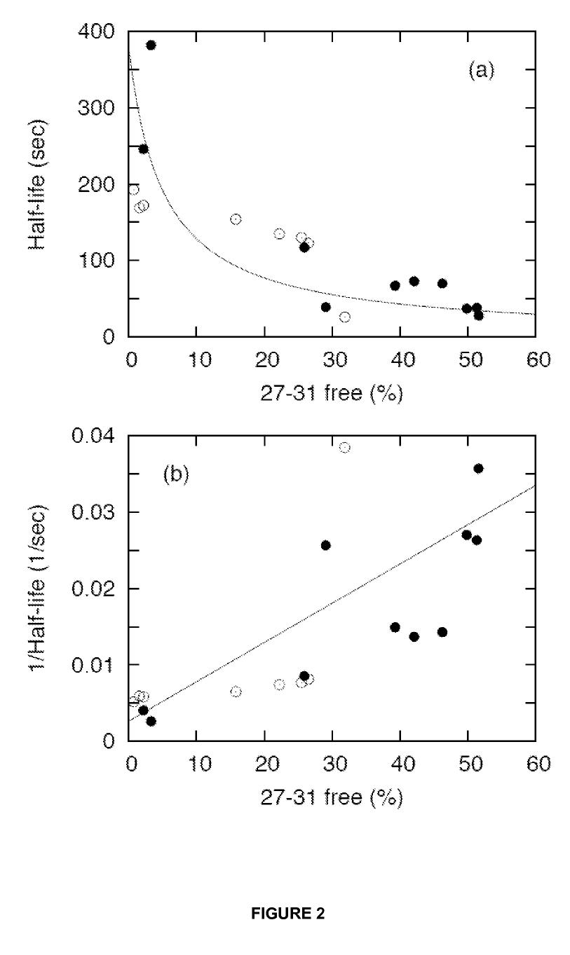

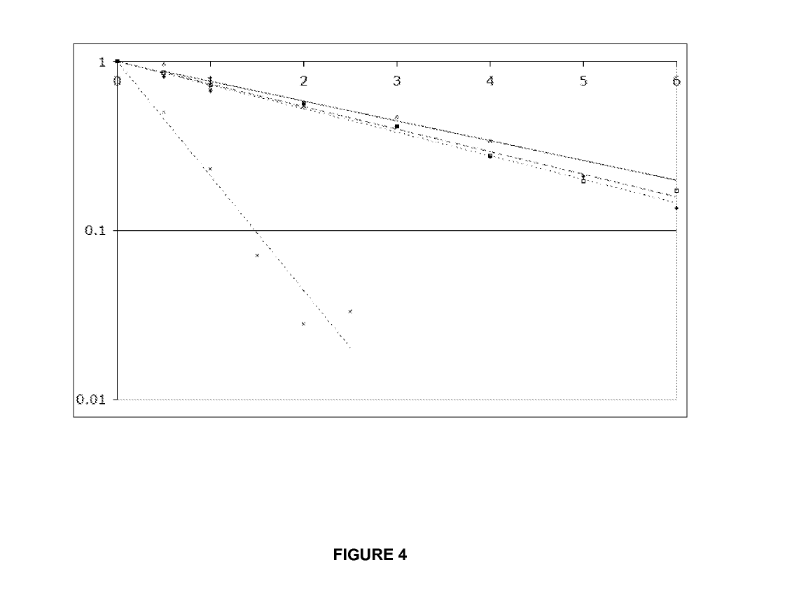

Typical read-outs from the applet for four of these lacZ variants are shown in FIG. 1. As seen from the figure all show large variations in the occupancy that result from the distribution of rapidly and slowly translated codons. Similar large variations in ribosome occupancy are also seen in most mRNAs where the codons in the early lacZ region were scrambled randomly (not shown). Examining these read-outs show that varying the initiation frequency or having different translation rates of the codons inserted between codon 5 and 10 in the wild type sequence does indeed affect the ribosome spacing further downstream as suggested by FIG. S1. Scrutiny of FIG. 1 reveals significant changes in the degree of occupancy up to about 50 codons from the sequence change after which the occupancy becomes the same. FIG. 1 also illustrates that the more stable mRNAs have a higher ribosome density on the initial part of the mRNA. In the right panels of FIG. 1, a window of five codons was moved down the mRNA and the fraction of time where these five codons were uncovered by ribosomes was estimated and plotted. For all ten lacZ variants and for the region from approximately codon 20 to codon 45, the inventors find a correlation between the fraction of time the 5 codons are uncovered and the mRNAs functional half-life. For other parts of the mRNA the correlation is not found (data not shown, but see FIG. 4). The best correlation the inventors find for the mRNA stretch from codon 27 to 31, and FIG. 2 show this for all ten mRNAs that were used to find the correlation.

The results indicate that ribosome occupancy of this early region of the mRNA should be of special importance for the mRNA half-life. Thus, the model predicts that insertion of slowly-translated codons before codon 20 in the wild type lacZ gene should decrease the functional stability because this specific region of the mRNA then would be less unoccupied by ribosomes. Similarly, insertions of slowly translated codons after codon 45 should increase the stability because ribosomes would form a queue behind these slowly translated codons and protect the region. These predictions were tested experimentally. As described in Methods, the inventors constructed the lacZ variants in pSN4 where the normal codons at position 16, 17 and 18 were exchanged with the slowly translated codons AGG CGG GGA.

Similarly, in the lacZ variants in pMAP210, pMAP211 and pMAP212 the normal codons at position 42; 42, 43, 44; or 42, 43, 44, 45 and 46 were replaced with the slowly translated codons AGG; AGG CGG GGA or AGG CGG GGA AGG CGG, respectively. Finally, the inventors constructed pMAPZZZ* and pMAPXXX where the stronger tufA Shine-Dalgarno sequenced from pIV1 replaced the normal lacZ Shine-Dalgarno region in pMAP211 and in pMAP212.

The wild type lacZ gene contains two slowly translated codons at position 31 and 32. In the three five variants with slowly translated codons inserted downstream of codon 42 the mRNA stability should be affected only slightly according to the model because it is difficult to create a bottleneck after another bottleneck. In order to distinguish the expected small changes in the mRNA half-lives, the inventors needed to improve the accuracy in the experiments. This was achieved by performing the half-life determinations on a mixture of two cultures: the lacZ variant to be tested and a lacZ reference variant. For each such experiment the time of sampling, temperature and other experimental conditions were therefore identical. As the internal reference the inventors used a culture contained a lacZ variant with an insert of 36 GAA codons at position 927 in lacZ, coding for a β-galactosidase protein with a higher molecular weight. As shown in FIG. S2 in the Supplementary Materials section, this allowed separation of the two β-galactosidase proteins by one-dimensional SDS gel electrophoresis.

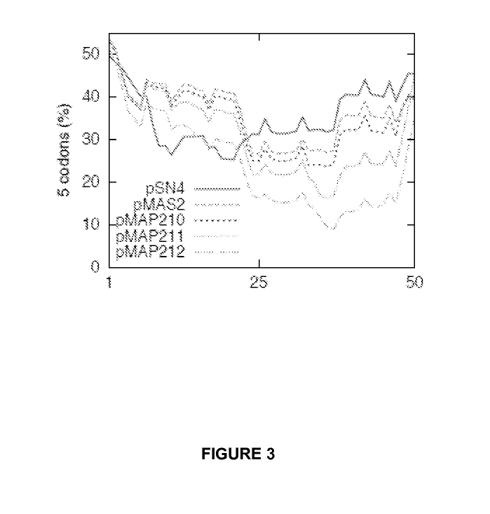

The functional half-life of these new lacZ mRNA variants was measured as previously described (Petersen, 1987) and shown in FIG. 3, normalized to the internal reference. The average half-life of the reference construct was 110 sec, identical to the wild-type lacZ mRNA half-life of 113 sec (Petersen, 1987). The inventors observed a considerable variation from experiment to experiment in the range of 93 sec to 116 sec for this reference mRNA and a similar variation for the other mRNAs. The early slowly translated codons in pSN4 destabilized the mRNA four-fold. Altering the sequence late in the coding region at codon 927 in pMAP217 was modelled to create a stretch of ribosome-free mRNA longer than that in pSN4 but as seen from FIG. 3 such distal ribosome-unoccupied mRNA region had little, if any influence on the mRNA functional half-life. In contrast, exchange of 1, 3 or 5 codons with slowly translated codons increased the mRNA functional half-life by approximately 5, 9 and 23%. Finally, the inventors modelled the ribosome occupancy along the mRNA for these five variants. FIG. 4 show that the occupancy in the region from codon 20 to 45 closely follows the functional half-life. Specifically, the ribosome-occupancy from codon 27 to 31 was calculated for the new lacZ variants and these results included in FIG. 2 (open circles). As seen, these measured functional half-lives correspond well to the predicted values. The reciprocal value of the experimentally determined half-lives and the fraction of time the mRNA from codon 27-31 were free, was plotted as shown in the supplementary materials FIG. S3. The inventors see that the points within experimental error now lie on a straight line extrapolating through (0,0) that is the mRNA fully covered with ribosomes and with an infinitely high half-life. If our model described the lacZ mRNA degradation only partially, this plot should not extrapolate to (0,0) because the additional degradation mechanism (s) would be active at a completely ribosome-covered mRNA.

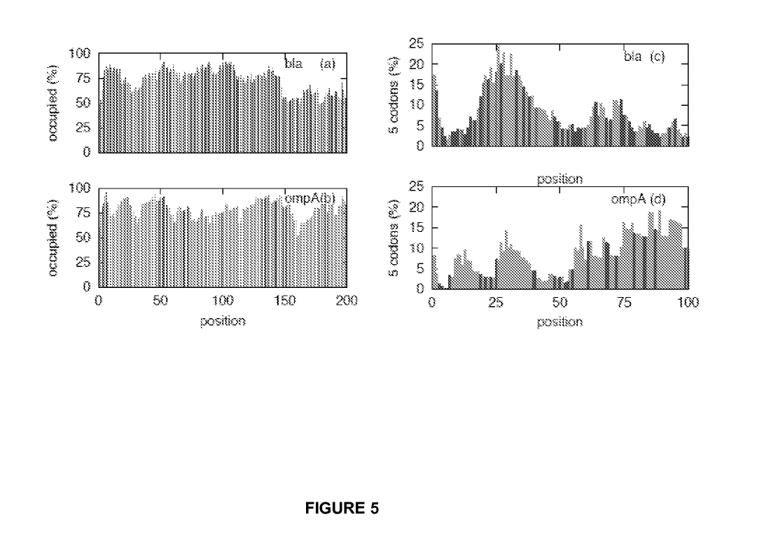

Finally, the inventors tried to see if our model had relevance for other mRNAs for which the functional half-life had been determined for instance for the OmpA and Lpp mRNAs that have an above average stability. However, the functional mRNA half-life of many membrane protein mRNAs is influenced by complex formation to small RNAs (Guillier et al 2006, Bossi and Figueroa-Bossi 2007). In the case of ompA the stability of the mRNA is modulated by binding of small RNA species to the untranslated 5′ end of the mRNA (Rasmussen et al, 2005). The proposed binding site is close to the ribosome-binding site and the binding of such small RNAs to the mRNA might therefore be influenced by the ribosome occupancy, but our model only describes occupancy in the translated part of the mRNA. However, mainly due to the strong Shine-Dalgarno interaction, the ompA mRNA should have a high density of ribosomes in its early coding region. The same holds for the rather stable bla mRNA (Nilsson et al 1984). With these caveats the inventors have modelled translation of the ompA and bla mRNA and the result shown in FIG. 5. Modelling the fraction of the time the codon 27*-31* mRNA is accessible by the applet for the bla and ompA mRNAs give values of between 10% and 20% which according to FIG. 2 would indicate mRNA half-lives above average for these two mRNAs in agreement with the experimental values.

Construction of Plasmids.

All the new lacZ variants were constructed by recombineering using single stranded oligoes with 35 base homologies on both sides of the sequence alteration using the plasmids pMAS2 or pIV1 (Sørensen and Pedersen, 1991, Vind et al 1993) as template and in E. coli HME70 essentially as described (Thomason et al., 2005; Sharan et al 2009). First a TAG stop codon was introduced in lacZ at position 13 or 42. After overnight incubation in rich medium at 30° C. with agitation, the culture was spread on plates containing 100 μg ampicilin and 40*μg 5-bromo-4-chloro-3-indolyl-b-D-galactopyranoside (X-Gal) per ml to screen for cells containing a defective lacZ gene on either pMAS2 or pIV1. White colonies were cross-streaked with phage Φ80supF that restores the activity of lacZ amber mutants. The presence of the TAG stop codon at the desired positions was verified by sequencing. The desired codon changes were again done by recombineering, screening for blue colonies on plates containing 100 μg ampicilin and 40*μg X-Gal per ml.

The plasmid pMAP217 with an insert of 36 GAA codons at position 927 in lacZ was constructed by first introducing an unique XhoI restriction site at position 927 by recombineering in lacZ on pMAS2. A 146 base long oligo containing thirty-six GAA codons was used to produce a double stranded DNA fragment with XhoI restriction site in both ends. The 146 base pair DNA fragment was cloned using the Zero Blunt TOPO PCR Cloning Kit (Invitrogen). The resulting plasmid was digested with XhoI and the 123 base pair XhoI DNA fragment was cloned in XhoI restricted pMAP201. The sequences of all plasmids constructed here are given in FIG. S2.

DNA Techniques.

Oligoes were supplied by DNA Technology A/S Denmark. Plasmid DNA was isolated using the Qiagen Plasmid kit. Eurofins MWG Operon, Germany performed DNA sequencing.

As mentioned above the present invention relates to DNA sequences containing mRNA stabilizing (or destabilizing) sequences and which upon transcription by a cell result in stabilized (or destabilized) mRNA transcripts, as well as to transformed microorganisms comprising such DNA sequences.

The use of such DNAs or stabilized mRNA transcripts in a method to increase the stability of mRNA transcripts of one or more genes that generate multiple mRNA transcripts and that are located on a chromosome, plasmid or any other self-replicating DNA molecule, or a method to increase the production of a desired chemical compound by a transformed microorganism, respectively, are objects of the present invention.

The term “cell” means a eukaryotic or prokaryotic cell.

The term “microorganism” means a microscopic, self-reproducing, respiring organism including, but not limited to, bacteria, fungi (including yeast) and algae. The term bacteria includes both Gram-negative and Gram-positive microorganisms. Examples of Gram negative bacteria are any from the genera Escherichia, Gluconobacter, Rhodobacter, Pseudomonas, and Paracoccus. Gram-positive bacteria are selected from, but not limited to any of the families Bacillaceae, Brevibacteriaceae, Corynebacteriaceae, Lactobacillaceae, and Streptococaceae and belong especially to the genera Bacillus, Brevibacterium, Corynebacterium, Lactobacillus, Lactococcus and Streptomyces. Among the genus Bacillus, B. subtilis, B. amyloliquefaciens, B. licheniformis and B. pumilus are preferred microorganisms in the context of the present invention. Among Gluconobacter, Rhodobacter and Paracoccus, G. oxydans, R. sphaeroides and P. zeaxanthinifaciens are preferred, respectively. Examples of yeasts are Saccharomyces, particularly S. cerevisiae. Examples of preferred other fungi are Aspergillus niger and Pencillium chrysogenum.

While the method of the present invention will be described in detail with respect to the expression of beta-galactosidase one skilled in the art will recognize that this method can be applied universally to increase the production of any protein to be synthesized by both prokaryotic (e.g. bacteria) and eukaryotic (e.g. fungi, plant and animal) cells.

### Example 1

By mathematical modelling, the inventors have analysed how the translation rate of individual codons influence the spacing of ribosomes on an mRNA. The inventors have focused on modelling ribosome trafficking in the early part of the coding region because breakdown of the mRNA takes place from the 5′ end (Jacquet and Kepes, 1971, Cannistraro and Kennell, 1985) and because sequence changes here affect the half-life (Petersen, 1987; Yarchuk et al 1992; Vind et al, 1993). The inventors found a clear correlation between the mRNAs functional half-life and the ribosome occupancy in the coding region of the mRNA from approximately codon 20 to 45.

The results presented in FIG. 2 were done analysing the occupancy of the mRNA from codon 27 to codon 31 that gives the best correlation to the mRNA half-life but other mRNA stretches as for example the stretch from codon 20 to 25 or from codon 25 to 40 give results that are only slightly different. However, it is only for this initial part of the coding region from approximately codon 20 to 45 such correlation can be observed, see FIG. 4.

As modelled previously (Mitarai et al 2008) there is a denser packing of the ribosomes early on the mRNA because of the higher density of slowly translated codons here (Bulmer 1988). Ribosomes initiate once per 2.3 seconds and physically cover about 11 codons. Therefore, the mRNA segment from codon 20 to 45 will often represent the space between the two ribosomes closest to the 5′ end of the mRNA at any time.

The degradosome model for degradation of mRNA (reviewed by Deana and Belasco, 2005) has the initial event being an endonucleolytic cut of the mRNA between two translating ribosomes as one of the options. The inventors do not think that such initial cut takes place for the following reason: If the cut were between the first two ribosomes, the ribosome preceding the cut would be expected to have its nascent peptide released as a tagged peptide by the tmRNA mechanism (Keiler et al 1996). The average mRNA is translated approximately 30 times (discussed by Mitarai et al 2008). A mechanism involving a cut between ribosomes would result in the release of 3% or more of all nascent peptides in the tagged unstable version. In addition to being wasteful, such release is an order of magnitude higher than the estimated amount of tagged peptides: 0.4% of the total number of nascent peptides (Moore and Sauer 2005).

The inventors therefore propose that the current model for mRNA degradation incorporate ribosome occupancy as follows: a component of the degradosome containing the RppH enzyme binds to an unoccupied part of the mRNA. Because slowly translated codons are overrepresented in the early part of the mRNA the distance from codon 20 to 45 are often free because ribosomes initiate 2.3 sec apart. Now, ribosome 1 releases the degrading enzyme complex in the proximity to the 5′ end. The degradosome will now either bind to a new target where it can not interact with a 5′-triphosphate group or the RppH enzyme will convert the nearby 5′ triphosphate to a mono-phosphate that destabilizes the mRNA (Celesnik et al 2007, Deana et al 2008). An interesting point in these speculations is whether the mRNA degradation machinery actually needs to be activated by a translating ribosome, in particular because the length of the 5′UTR and mRNA stability seem not to correlate and because other cellular RNA with exposed 5′ mono-phosphate groups as for example tRNA are normally very stable. Evidently and unfortunately, modelling cannot elucidate such specific biochemical mechanisms.

Because the inventors have mainly modelled mRNAs that are almost identical the inventors cannot exclude that additional parameters such as the mRNA length, sequence and structure also influences the stability.

Our analysis of the ribosome spacing is dependent on a correct estimate of the resulting on-rate. As mentioned above, the plasmids used by Petersen, (1987) all had the same Shine-Dalgarno sequence and the same first five codons in the coding region. For the plasmids pIV1 and pIV18 with a presumed higher affinity for the initiating 30S ribosome, the inventors tested the robustness of our determination of the spacing by using resulting on-rates that were two-fold above and two-fold below the values the inventors have used, estimated as described by Freier et al (1987). These results are indicated on FIG. S3 (open triangle symbols***). As seen, these up to four-fold changes in the on-rates had only a minor influence on the modelled ribosome spacing on the first part of the mRNA and did only slightly change the correlation between the half-life and the fraction of the time this mRNA stretch is accessible.

In most other cases, it is not possible to investigate whether the more stable natural mRNAs are more occupied by ribosomes compared to the unstable natural mRNAs because the inventors lack information about on-rate for translation initiation or about the functional half-lives. Modelling of the natural mRNAs for which the inventors previously had determined the half-life (Pedersen et al 1978) is also difficult because these experiments were carried out in an E. coli B strain, with a yet incompletely sequenced genome and where the concentration of initiation-competent ribosomes might be different. Furthermore, many of the functional half-lives determined in this study were ribosomal protein mRNAs where translational coupling ensures that the on-rate for translating these mRNAs cannot be calculated directly from the Shine-Dalgarno interaction. The interaction between small regulatory RNAs and the initiation region (Bossi and Figueroa-Bossi, 2007) also makes it difficult to evaluate if for instance the functional mRNA half-lives determined by Yarchuk et al, (1992) are as predicted by our model.

The study of Ringquist et al, (1992) provided a detailed study of how varying the Shine-Dalgarno interaction affected lacZ expression. However, no functional half-life was measured directly in this study. It is therefore not known if the observed effects on lacZ expression were because of an altered on-rate for translation initiation, an altered on-rate that changed the mRNA half-life, or an altered transcriptional polarity. These data are not in contradiction to our model because as found for the pIV1 and pIV18 mRNAs and for the mRNAs it is very likely that they resulted from an altered on-rate that changed the mRNA half-life via an influence on the ribosome spacing.

Several examples are known where a specific mRNA sequence has an effect on the mRNA half-life. One example of this is the finding that a ribosomal protein S1-binding AU rich mRNA sequence can stabilize an mRNA (Komarova et al (2005). According to our modelling, the mechanism behind the mRNA stabilization of this sequence might well be that avid binding to ribosomal protein S1 to such mRNA sequence increases the on-rate for 30S ribosome binding and that this decreases the ribosome spacing and increases the mRNA half-life.

Mitarai et al, (2008) found that the preponderance of slowly translated codons in the 5′ end of the mRNA was a highly conserved feature and suggested that this conservation had to do with fine-tuning the translation initiation frequency or had importance for the overall ribosome efficiency. In addition, our modelling suggests that the conserved codon usage in the early part of the mRNA via differences in the translation rate of the individual codons also has evolved to provide the mRNA with a suitable functional half-life. It is a common observation that the activity of an enzyme often is insensitive to amino acid changes in the N-terminus. The β-galactosidase protein is a well-known example of this where up to the 41 N-terminal amino acids can be changed (Brickman et al, 1979) and where a plethora of fusion proteins to 5′ end of lacZ still retain enzyme activity. Also it is commonly observed that various amino acid sequences, for instance a his-tag can be added to the N-terminus of various enzymes without disturbing the function of the protein. It is therefore conceivable that genes frequently have close to total freedom to evolve N-termini with an amino acid usage and codon usage that results in a suitable mRNA half-life.

Finally, the inventors note that the distance between translating ribosomes in specific regions of the mRNA may be rate determining for degradation for at least some eukaryotic mRNAs (Lemm and Ross 2002). The mechanism in this study involved binding of proteins to the mRNA but even so, local translation rate differences may be a mechanism for governing the accessibility of components that affects mRNA degradation in all organisms.

### RE EXAMPLES 2 & 3

In the below discussed Examples 2 and 3 stabilized/destabilized GFP mRNA variants are designed for expression in either CHO cells (Example 2) and Bacillus (Example 3).

The Examples aim to support the concept of the present invention, namely, to alter codons either before codon 20 or immediately after codon 45 in such a way that codons 20-45 of the mRNA region become either more or less covered with ribosomes. This will stabilize or destabilize the mRNA. To stabilize the mRNA the codon changes should make the codons immediately after codon 45 slower translated compared to the wild type reference; to further stabilize the mRNA the codons before codon 20 may be faster translated. To destabilize the mRNA the codon changes should make the codons before codon 20 slower translated; to further destabilize the mRNA the codons after codon 45 may be faster translated. Also, the codons in the region 20-45 may be changed to faster codons in the case where a mRNA should be destabilized to remove possible ribosome queues in this region.

In the case Bacillus subtilis (cf Example 3) the codon usage in highly expressed genes is shown in Table 1.

TABLE 1

| | | | | | | | |
|---|---|---|---|---|---|---|---|
| TTT phe F | 41 | TCT ser S | 160 | TAT tyr Y | 36 | TGT cys C | 7 |
| TTC phe F | 102 | TCC ser S | 11 | TAC tyr Y | 104 | TGC cys C | 8 |
| TTA leu L | 107 | TCA ser S | 63 | TAA OCH * | 43 | TGA OPA * | — |
| TTG leu L | 56 | TCG ser S | 2 | TAG AMB * | 2 | TGG trp W | 26 |
| CTT leu L | 201 | CCT pro P | 97 | CAT his H | 40 | CGT arg R | 291 |
| CTC leu L | 11 | CCC pro P | 5 | CAC his H | 61 | CGC arg R | 141 |
| CTA leu L | 37 | CCA pro P | 91 | CAA gln Q | 142 | CGA arg R | 7 |
| CTG leu L | 26 | CCG pro P | 27 | CAG gln Q | 33 | CGG arg R | 1 |
| ATT ile I | 152 | ACT thr T | 168 | AAT asn N | 56 | AGT ser S | 20 |
| ATC ile I | 226 | ACC thr T | 5 | AAC asn N | 195 | AGC ser S | 45 |
| ATA ile I | 3 | ACA thr T | 112 | AAA lys K | 528 | AGA arg R | 38 |
| ATG met M | 145 | ACG thr T | 44 | AAG lys K | 115 | AGG arg R | 4 |
| GTT val V | 264 | GCT ala A | 284 | GAT asp D | 130 | GGT gly G | 241 |
| GTC val V | 51 | GCC ala A | 23 | GAC asp D | 111 | GGC gly G | 93 |
| GTA val V | 175 | GCA ala A | 147 | GAA glu E | 340 | GGA gly G | 158 |
| GTG val V | 50 | GCG ala A | 68 | GAG glu E | 103 | GGG gly G | 10 |

In the case of CHO cells (cf Example 2) the codon usage in highly expressed genes is shown in Table 2.

TABLE 2

| | | | | | | | |
|---|---|---|---|---|---|---|---|
| TTT phe F | 9 | TCT ser S | 11 | TAT tyr Y | 7 | TGT cys C | 5 |
| TTC phe F | 7 | TCC ser S | 7 | TAC tyr Y | 11 | TGC cys C | 1 |
| TTA leu L | 2 | TCA ser S | 3 | TAA OCH * | 1 | TGA OPA * | 1 |
| TTG leu L | 5 | TCG ser S | 2 | TAG AMB * | — | TGG trp W | 7 |
| CTT leu L | 6 | CCT pro P | 13 | CAT his H | 7 | CGT arg R | 12 |
| CTC leu L | 4 | CCC pro P | 8 | CAC his H | 8 | CGC arg R | 4 |
| CTA leu L | 2 | CCA pro P | 12 | CAA gln Q | 2 | CGA arg R | 3 |
| CTG leu L | 26 | CCG pro P | — | CAG gln Q | 12 | CGG arg R | 2 |
| ATT ile I | 20 | ACT thr T | 13 | AAT asn N | 8 | AGT ser S | 5 |
| ATC ile I | 16 | ACC thr T | 13 | AAC asn N | 10 | AGC ser S | 7 |
| ATA ile I | 3 | ACA thr T | 8 | AAA lys K | 28 | AGA arg R | 7 |
| ATG met M | 14 | ACG thr T | — | AAG lys K | 37 | AGG arg R | 3 |
| GTT val V | 17 | GCT ala A | 32 | GAT asp D | 16 | GGT gly G | 22 |
| GTC val V | 15 | GCC ala A | 12 | GAC asp D | 17 | GGC gly G | 18 |
| GTA val V | 6 | GCA ala A | 5 | GAA glu E | 13 | GGA gly G | 10 |
| GTG val V | 16 | GCG ala A | 3 | GAG glu E | 19 | GGG gly G | 2 |

The above principles and these two tables were then used to suggest codon changes that would stabilize (Example 2), respectively destabilize (Example 3) the GFP mRNA in these two expression systems, CHO and Bacillus, respectively.

### Example 2

### eGFP Analysis in CHO Cells

Genetic Constructions for eGFP Expression in CHO Cells.

Three different eGFP genes were designed. These are an unmodified eGFP gene (SEQ ID NO 1), a gene leading to stabilized mRNA (SEQ ID NO 4), and a gene leading to destabilized mRNA (SEQ ID NO 5). All genes were synthesized, and sequenced, by Geneart. They contain a 5′ HindIII-site and a 3′ XhoI-site, which was used for cloning in pcDNA4/TO from the T-REx system from Invitrogen (Carlsbad, Calif.).

Resulting plasmids were partly sequenced after cloning to confirm that the cloning region sequence were as predicted. Large scale plasmid preparations were made using an EndoFree Plasmid Mega kit from Qiagen (Hilden, Germany).

The GFP Wild type sequence (SEQ ID NO 1) has the following sequence:

atg agt aaa gga gaa gaa ctt ttc act gga gtt gtc cca att ctt gtt gaa tta gat ggt gat gtt aat ggg
cac aaa ttt tct gtc agt gga gag ggt gaa ggt gat gca aca tac gga aaa ctt acc ctt aaa ttt att tgc
act act gga aaa cta cct gtt cca tgg cca aca ctt gtc act act ttc ggt tat ggt gtt caa tgc ttt gcg aga
tac cca gat cat atg aaa cag cat gac ttt ttc aag agt gcc atg cct gaa ggt tat gta cag gaa aga act
ata ttt ttc aaa gat gac ggg aac tac aag aca cgt gct gaa gtc aag ttt gaa ggt gat acc ctt gtt aat
aga atc gag tta aaa ggt att gat ttt aaa gaa gat gga aac att ctt gga cac aaa ttg gaa tac aac tat
aac tca cac aat gta tac atc atg gca gac aaa caa aag aat gga atc aaa gtt aac ttc aaa att aga
cac aac att gaa gat gga agc gtt caa cta gca gac cat tat caa caa aat act cca att ggc gat ggc
cct gtc ctt tta cca gac aac cat tac ctg tcc aca caa tct gcc ctt tcg aaa gat ccc aac gaa aag aga
gac cac atg gtc ctt ctt gag ttt gta aca gct gct ggg att aca cat ggc atg gat gaa cta tac aaa taa

The GFP modified for the mRNA being more stable (SEQ ID NO 4) has the following sequence, wherein base changes compared to the wild type are shown in upper case font:

atg agt aaa gga gaa gaa ctG ttc act gga gtt gtc cca att ctG gtt gaa CTG gat ggt gat gtt aat
ggT cac aaa ttt tct gtc agt gga gag ggt gaa ggt gat gca aca tac gga aaa ctt acc ctt aaa ttt att
tgc act act ggG aaa cta ccC gtA ccG tgg ccC acG ctA gtc act act ttc ggG tat ggG gtA caa tgc
ttt gcg agG tac cca gat cat atg aaa cag cat gac ttt ttc aag agt gcc atg cct gaa ggt tat gta cag
gaa aga act ata ttt ttc aaa gat gac ggg aac tac aag aca cgt gct gaa gtc aag ttt gaa ggt gat
acc ctt gtt aat aga atc gag tta aaa ggt att gat ttt aaa gaa gat gga aac att ctt gga cac aaa ttg
gaa tac aac tat aac tca cac aat gta tac atc atg gca gac aaa caa aag aat gga atc aaa gtt aac
ttc aaa att aga cac aac att gaa gat gga agc gtt caa cta gca gac cat tat caa caa aat act cca att
ggc gat ggc cct gtc ctt tta cca gac aac cat tac ctg tcc aca caa tct gcc ctt tcg aaa gat ccc aac
gaa aag aga gac cac atg gtc ctt ctt gag ttt gta aca gct gct ggg att aca cat ggc atg gat gaa cta
tac aaa taa

The GFP modified for the mRNA being more unstable (SEQ ID NO 5) has the following sequence, wherein base changes compared to the wild type are shown in upper case font:

atg agt aaa gga gaa gaa ctt ttc act ggG gtt gtc ccG att ctA gtA gaa tta gat ggG gat gtA aat
ggg cac aaa ttt tct gtc agt gga gag ggt gaa ggt gat gcT aca tac gga aaa ctG acc ctG aaa ttt
att tgc act act ggT aaa ctG cct gtt cca tgg cca aca ctG gtc act act ttc ggt tat ggt gtt caa tgc ttt
gcg aga tac cca gat cat atg aaa cag cat gac ttt ttc aag agt gcc atg cct gaa ggt tat gta cag gaa
aga act ata ttt ttc aaa gat gac ggg aac tac aag aca cgt gct gaa gtc aag ttt gaa ggt gat acc ctt
gtt aat aga atc gag tta aaa ggt att gat ttt aaa gaa gat gga aac att ctt gga cac aaa ttg gaa tac
aac tat aac tca cac aat gta tac atc atg gca gac aaa caa aag aat gga atc aaa gtt aac ttc aaa
att aga cac aac att gaa gat gga agc gtt caa cta gca gac cat tat caa caa aat act cca att ggc
gat ggc cct gtc ctt tta cca gac aac cat tac ctg tcc aca caa tct gcc ctt tcg aaa gat ccc aac gaa
aag aga gac cac atg gtc ctt ctt gag ttt gta aca gct gct ggg att aca cat ggc atg gat gaa cta tac
aaa taa

### Transient Gene Expression Experiments.

### Expression Experiment 1:

Plasmid pcDNA4/TO-derivatives were used to transfect CHO cells using the “FreeStyle MAX CHO expression System. These plasmids contain the TetO₂ operator, enabling regulated expression when TetR repressor is present. Since this repressor is not present in CHO FreeStyle cells, gene expression will take place in a constitutive fashion, from introduction of the plasmid (transfection), and until the plasmid is lost from culture (due to lack of replication). As a negative control pcDNA4/TO was included in the experiment. During the experiment care was taken to ensure that exactly the same amount of plasmid was used in all four cases (negative control, wild type, stabilized and destabilized), and that all four cultures were treated in parallel and exactly the same way. Samples were extracted from cultures at the day of transfection and the five following days.

Samples were used for cell counting, GFP measument using “GFP Quantification Kit, Fluorometric” from Cell Biolabs Inc. (San Diego, Calif.), and for Real-time RT-PCR (as below) on selected samples.

### Expression Experiment 2:

This was carried out as described for experiment 1 with the following exceptions: The plasmid pcDNA6/TR was included in six fold excess in all transfections, as described in the instructions for the T-REx system. pcDNA6/TR encodes the TetO₂ operator, and, consequently, expression only takes place from the pcDNA4/TO-derivatives, when the inducer, tetracycline, is added to the culture. Also, in this experiment a positive control plasmid (pcDNA4/TO/lacZ) was included, and finally, two cultures were set up for each plasmid.

After transfection, cultures were allowed to grow one day before tetracycline was added (to 1 μg/mL). After one more day, tetracycline was removed by media change. Culture samples were extracted from transfection and until day five.

### Results

eGFP Protein Quantification

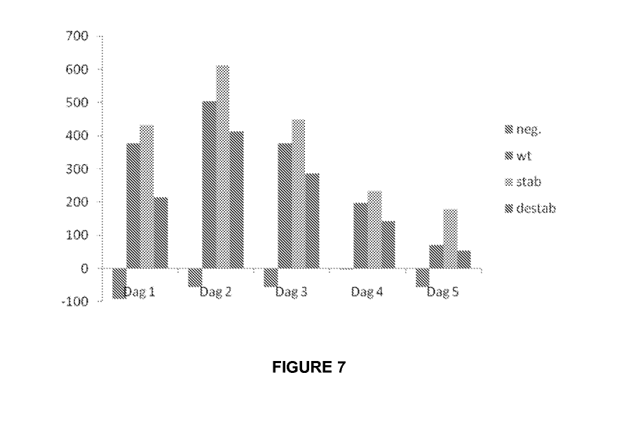

The results of GFP quantification from experiment 1 is shown in FIG. 7. The results are perfectly in agreement the expected results (highest yield for the stabilized and lowest yield for the destabilized construct, for all five days).

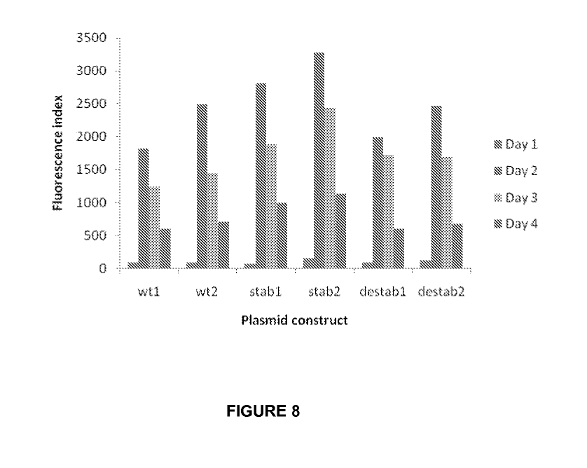

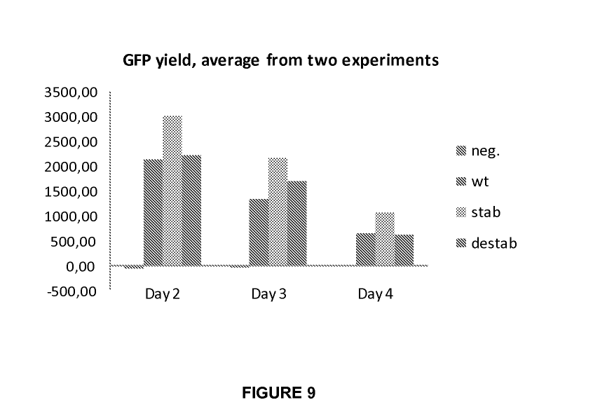

The results of GFP quantification form samples from experiment 2 are shown in FIG. 8 and FIG. 9.

### Example 3

### eGFP Analysis in Bacillus subtilis

eGFP Genes for Bacillus subtilis Expression.

The first gene encoded the wild type eGFP sequence (SEQ ID NO 1), the second gene encoded an eGFP gene having a stabilized eGFP mRNA (SEQ ID NO 2), and the third gene encoded an eGFP gene having a destabilized eGFP mRNA (SEQ ID NO 3).

The GFP Wild type sequence (SEQ ID NO 1) has the following sequence:

atg agt aaa gga gaa gaa ctt ttc act gga gtt gtc cca att ctt gtt gaa tta gat ggt gat gtt aat ggg
cac aaa ttt tct gtc agt gga gag ggt gaa ggt gat gca aca tac gga aaa ctt acc ctt aaa ttt att tgc
act act gga aaa cta cct gtt cca tgg cca aca ctt gtc act act ttc ggt tat ggt gtt caa tgc ttt gcg aga
tac cca gat cat atg aaa cag cat gac ttt ttc aag agt gcc atg cct gaa ggt tat gta cag gaa aga act
ata ttt ttc aaa gat gac ggg aac tac aag aca cgt gct gaa gtc aag ttt gaa ggt gat acc ctt gtt aat
aga atc gag tta aaa ggt att gat ttt aaa gaa gat gga aac att ctt gga cac aaa ttg gaa tac aac tat
aac tca cac aat gta tac atc atg gca gac aaa caa aag aat gga atc aaa gtt aac ttc aaa att aga
cac aac att gaa gat gga agc gtt caa cta gca gac cat tat caa caa aat act cca att ggc gat ggc
cct gtc ctt tta cca gac aac cat tac ctg tcc aca caa tct gcc ctt tcg aaa gat ccc aac gaa aag aga
gac cac atg gtc ctt ctt gag ttt gta aca gct gct ggg att aca cat ggc atg gat gaa cta tac aaa taa

The GFP modified for the mRNA being more stable (SEQ ID NO 2) has the following sequence, wherein base changes compared to the wild type are shown in lower case font:

ATG AGc AAA GGA GAA GAA CTT TTC ACT GGA GTT GTt CCA ATT CTT GTT GAA TTA
GAT GGT GAT GTT AAc GGt CAC AAA TTT TCT GTC AGT GGA GAG GGT GAA GGT
GAT GCA ACA TAC GGA AAA CTT ACC CTT AAA TTT ATT TGC ACc ACg GGg AAg CTA
CCc GTc CCc TGG CCc ACc CTT GTC ACc ACg TTC GGT TAT GGT GTT CAA TGC TTT
GCG AGA TAC CCA GAT CAT ATG AAA CAG CAT GAC TTT TTC AAG AGT GCC ATG
CCT GAA GGT TAT GTA CAG GAA AGA ACT ATA TTT TTC AAA GAT GAC GGG AAC
TAC AAG ACA CGT GCT GAA GTC AAG TTT GAA GGT GAT ACC CTT GTT AAT AGA
ATC GAG TTA AAA GGT ATT GAT TTT AAA GAA GAT GGA AAC ATT CTT GGA CAC
AAA TTG GAA TAC AAC TAT AAC TCT CAC AAT GTA TAC ATC ATG GCA GAC AAA
CAA AAG AAT GGA ATC AAA GTT AAC TTC AAA ATT AGA CAC AAC ATT GAA GAT
GGA AGC GTT CAA CTA GCA GAC CAT TAT CAA CAA AAT ACT CCA ATT GGC GAT
GGC CCT GTC CTT TTA CCA GAC AAC CAT TAC CTG TCC ACA CAA TCT GCG CTT
TCG AAA GAT CCC AAC GAA AAG AGA GAC CAC ATG GTC CTT CTT GAG TTT GTA
ACA GCT GCT GGG ATT ACA CAT GGC ATG GAT GAA CTA TAC AAA TAA

The GFP modified for the mRNA being more unstable (SEQ ID NO 3) has the following sequence, wherein base changes compared to the wild type are shown in upper case font:

atg agt aaa gga gaa gaa ctt ttc act gga gtC gtc ccC att ctG gtt gaG tta gat ggt gat gtt aaC
ggT cac aaa ttC tct gtT agC ggT gaA ggt gaa ggt gat gca aca tac gga aaa ctt acT ctt aaa ttt
att tgc act act ggT aaa ctT cct gtt cca tgg cca aca ctt gtc act act ttc ggt tat ggt gtt caa tgc ttt
gcg aga tac cca gat cat atg aaa cag cat gac ttt ttc aag agt gcc atg cct gaa ggt tat gta cag gaa
aga act ata ttt ttc aaa gat gac ggg aac tac aag aca cgt gct gaa gtc aag ttt gaa ggt gat acc ctt
gtt aat aga atc gag tta aaa ggt att gat ttt aaa gaa gat gga aac att ctt gga cac aaa ttg gaa tac
aac tat aac tca cac aat gta tac atc atg gca gac aaa caa aag aat gga atc aaa gtt aac ttc aaa
att aga cac aac att gaa gat gga agc gtt caa cta gca gac cat tat caa caa aat act cca att ggc
gat ggc cct gtc ctt tta cca gac aac cat tac ctg tcc aca caa tct gcc ctt tcg aaa gat ccc aac gaa
aag aga gac cac atg gtc ctt ctt gag ttt gta aca gct gct ggg att aca cat ggc atg gat gaa cta tac
aaa taa

All three genes were synthesized by Geneart (Germany) and contained 5′ BamHI and 3′ SmaI restriction sites for cloning into the IPTG inducible gene expression pHT01 from MoBiTec (Germany) (www.mobitec.com). All three eGFP genes have been sequenced as part of the quality control at Geneart.

The three genes from Geneart have the following numbers:

Geneart No 1106690; eGFP wild type for Bacillus subtilis
Geneart No 1106691; eGFP stabilized for Bacillus subtilis
Geneart No 1106692; eGFP destabilized for Bacillus subtilis
Cloning of eGFP Genes into Bacillus subtilis Expression Vector pHT01

The eGFP genes were excised from the plasmids obtained from Geneart and inserted into the BamHI/SmaI sites of the expression vector pHT01 using standard cloning procedures. The vector pHT01 is an E. coli-B. subtilis shuttle vector that allows high-level expression of recombinant proteins within the cytoplasm. The expression vector uses the strong GA-dependent promoter preceding the groESL operon of B. subtilis fused to the lac operator allowing the induction by addition of IPTG.

The ligation mixture was transformed into E. coli DH10B electro competent cells and transformants were selected on LB-agar plates containing 100 mg/l of ampicillin. Transformants containing the expected recombinant plasmids were identified by colony PCR using the two primers pHT01 P1 forward: (5′ GGGAGCGGAAAAGAATGATGTAAGCGTG 3′) and pHT01 P2 reverse: (5′ GACAAAGATCTCCATGGACGCGTGACGTG 3′). One clone from each transformation showing the expected PCR product was isolated, re-streaked and stored in glycerol as research Master Cell Bank (rMCB) with the following numbers:

UP1036; pHT01::eGFP wt/DH10B
UP1037; pHT01::eGFP stabilized/DH10B
UP1038; pHT01::eGFP destabilized/DH10B

Plasmid DNA was purified from strains UP1036, UP1037 and UP1038 using the JetStar Midiprep purification kit (Genomed, Germany). The recombinant plasmids were verified by restriction enzyme digestion and by DNA sequencing of the cloning junctions using the two primers pHT01 P1 forward and pHT01 P2 reverse. Both analyses confirmed the correct insertion of the three eGFP variants into the pHT01 vector.

Transformation of Bacillus subtilis Strain MT102

Each of the three plasmids were subsequently transformed into B. subtilis MT102 (strain provided by MoBiTec) using the transformation protocol supplied by MoBiTec. Selection was performed on LB-agar plates containing 5 mg/l of chloramphenicol. Two clones from each transformation were re-streaked and stored in glycerol as research Master Cell Bank (rMCB) with the strain numbers below. As control we transformed pHT01 into B. subtilis strain MT102 as well.

UP1032; pHT01/MT102
UP1043; pHT01::eGFP wt/MT102 clone 1
UP1044; pHT01::eGFP wt/MT102 clone 2
UP1045; pHT01::eGFP stabilized/MT102 clone 1
UP1046; pHT01::eGFP stabilized/MT102 clone 2
UP1047; pHT01::eGFP destabilized/MT102 clone 1
UP1048; pHT01::eGFP destabilized/MT102 clone 2

For the analysis of eGFP expression the four strains UP1032, UP1043, UP1045 and UP1047 were used.

### Induction Experiment 1

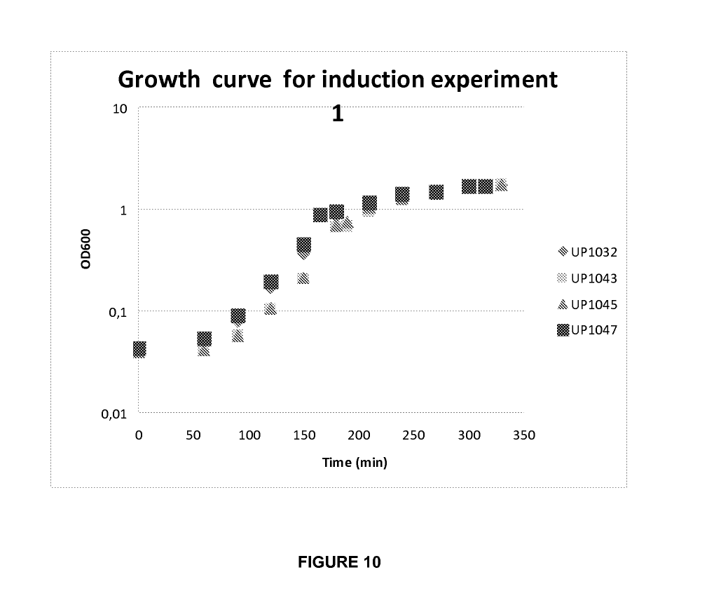

The four strains UP1032, UP1043, UP1045 and UP1047 were grown overnight in 10 ml LB medium containing 5 mg/L of chloramphenicol at 37° C. The overnight cultures were diluted 100 fold in 100 ml fresh medium and grown (shaking 250 rpm) until OD600≈0.7-0.8, where the cultures were induced using IPTG (final concentration 1 mM). Samples (2×2.5 ml, 2×5 ml, 2×10 ml) were harvested after 2½ hours of IPTG induction. FIG. 10 shows the growth curve for this experiment.

Protein lysates were prepared using FastPrep FP120 equipment as shortly described below. The cell pellets were washed in 1 ml 1× lysis buffer (supplied in the GFP quantification kit (AKR120 from CELL BIOLABS INC), centrifuged, re-suspended in 200 μl 1× lysis buffer and then transferred to a new tube (with screw cap) containing acid washed glass beads (107 micron, SIGMA). The cell suspension was treated in the FastPrep for 25 seconds at max speed (6.5), and then rested for 1 minute on ice. This procedure was repeated three times in total. Another 150 μl 1× lysis buffer was added to the tube and the supernatants (ca 350 μl) containing the soluble protein fractions were obtained by centrifugation.

### Induction Experiment 2

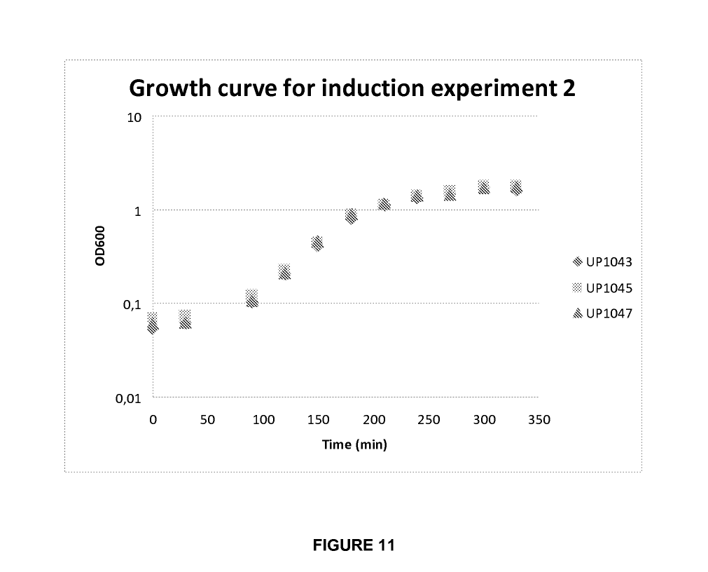

In this experiment the negative control strain UP1032 was omitted. This induction experiment was executed as the first experiment with few exceptions; Cultures were induced at OD600≈0.8-0.9. FIG. 11 shows the growth curve for this experiment. Only one set of cell extract preparations was performed in this experiment.

SDS-PAGE Analysis of eGFP Expression

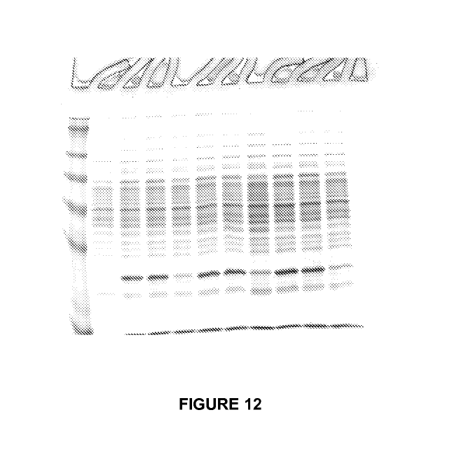

The protein lysates were analyzed by SDS-PAGE (12% Tris-Glycin) in order to visualize the expression of eGFP. 10 μl protein lysdate was mixed with 10 μl sample buffer and loaded on the SDS-PAGE. The SDS-PAGE clearly demonstrates the expression of a recombinant protein having the expected molecular weight of eGFP (26.8 KDa). No expression is seen in the negative control lysate (UP1032; lane 2). Expression is very similar in UP1043 (wild type eGFP; lane 3) and in UP1045 (stabilized eGFP; lane 4), while the expression in UP1047 (destabilized eGFP; lane 5) is much lower (FIG. 12). This pattern is independent of the two induction experiments and independent of the two protein extractions performed for the first induction experiment.

Fluorometric Quantification of eGFP

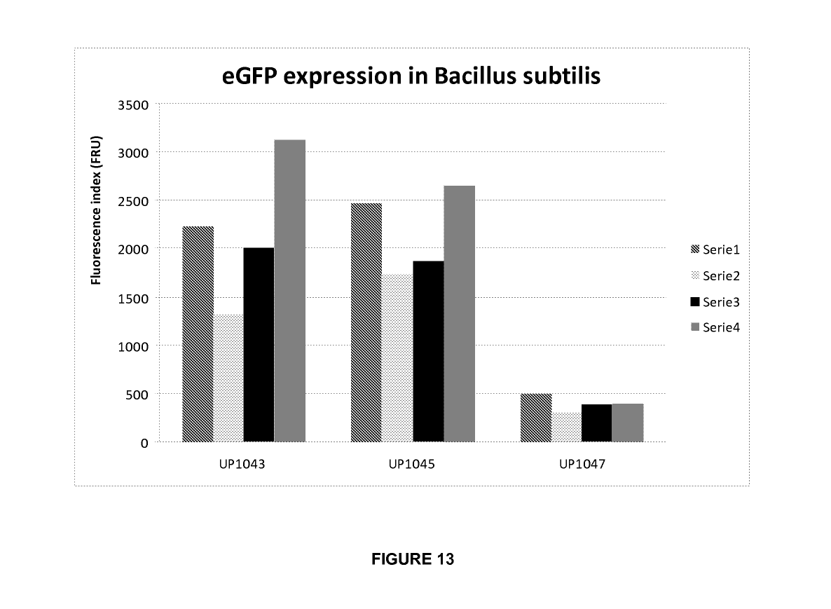

The expression of eGFP in the different expression constructs were quantified using the GFP Quantification Kit from CELL BIOLABS INC (Cat. Number AKR 120). The procedure and assay protocol were followed as described by the manufacturer of the kit. Generally the samples were diluted 10 times in lysis/assay buffer in order to be within the range of the standard curve. The fluorescence was measured using a fluorescence plate reader at 485/538 nm. Each sample was analyzed in duplicate in the plate reader. The relative fluorescence is shown in FIG. 13.

The figure shows that the fluorescence in UP1047 (destabilized eGFP) is 4-8 times lower than the level of fluorescence in the wild type or stabilized strains (UP1043 and UP1045). The first extraction performed on the cells from induction experiment 1 showed that the stabilized eGFP variant resulted in approximately 10% higher fluorescence compared to the wild type variant; comparison of the green and red bars in strains UP1043 and UP1045. However, when the experiment was repeated using the second extract from the first induction experiment and an extract from the second induction experiment, the results were somehow inverted. Here, the fluorescence in the strain containing the wild type eGFP gene was approximately 5-15% higher than the stabilized variant; comparison of the blue and yellow bars in strains UP1043 and UP1045.

### Isolation of Total RNA

Total RNA was isolated from the 10 ml cell pellets (UP1032, UP1043, UP1045, and UP1047) obtained from induction experiment 1. The Qiagen RNeasy Midi Kit was used according the instructions from the manufacturer (Handbook September 2010). Total RNA of high purity were obtained from all four strains. The specifications for the RNA are given in table 3.

TABLE 3

| Strain | Concentration | A260/A280 | Total amount (μg) |
|---|---|---|---|
| UP1032 | 134 ng/μl | 2.1 | 33.5 μg |
| UP1043 | 110 ng/μl | 2.1 | 27.5 μg |
| UP1045 | 88 ng/μl | 2.1 | 22.0 μg |
| UP1047 | 85 ng/μl | 2.1 | 21.2 μg |

Quantification of eGFP mRNA with qPCR Analysis

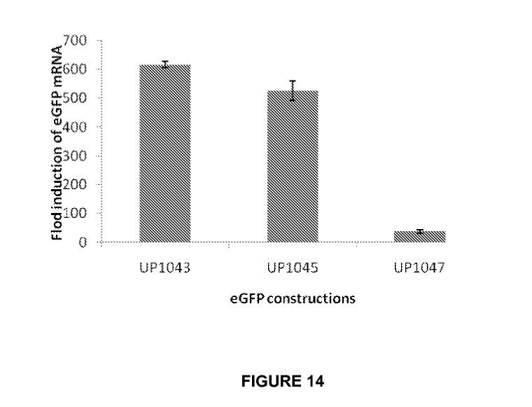

The mRNA levels of eGFP in the different expression constructs were quantified using Real-time RT-PCR (qPCR). The protocol from Applied Biosystems was followed as described in Tag Man® RNA-to-C₁TM 1-Step Kit Part No. 4392938. GFP specific primers for qPCR analysis were supplied from Applied Biosystems. FIG. 14 shows qPCR analysis of eGFP mRNA levels in B. subtilis. Fold induction normalized to control cultures.

### REFERENCES

Apirion D (1973) Degradation of RNA in Escherichia coli. A hypothesis. Mol Gen Genet; 122: 313-22.
Babitzke P, Kushner S R (1991) The Ams (altered mRNA stability) protein and ribonuclease E are encoded by the same structural gene of Escherichia coli. Proc Natl Acad Sci USA; 88: 1-5.
Blundell M, Craig E, Kennell D (1972) Decay rates of different mRNA in E. coli and models of decay. Nat New Biol; 238: 46-9.
Bossi L, Figueroa-Bossi N (2007) A small RNA downregulates LamB maltoporin in Salmonella. Mol Microbiol; 65: 799-810.
Brickman E, Silhavy T J, Bassford P J Jr, Shuman H A, Beckwith J R (1979) Sites within gene lacZ of Escherichia coli for formation of active hybrid beta-galactosidase molecules. J Bacteriol; 139: 13-8.
Bulmer M (1988) Codon usage and intragenic position. J Theor Biol; 133:67-71.
Carabetta V J, Mohanty B K, Kushner S R, Silhavy T J (2009) The response regulator SprE (RssB) modulates polyadenylation and mRNA stability in Escherichia coli. J. Bacteriol; 191: 6812-21.
Celesnik H, Deana A, Belasco J G (2007) Initiation of RNA decay in Escherichia coli by 5′ pyrophosphate removal. Mol Cell; 27: 79-90.
Cheng Z F, Deutscher M P (2005) An important role for RNase R in mRNA decay. Mol Cell; 17: 313-8.
Cannistraro V J, Kennell D (1985) Evidence that the 5′ end of lac mRNA starts to decay as soon as it is synthesized. J Bacteriol; 161: 820-2.
Deana A, Celesnik H, Belasco J G (2008) The bacterial enzyme RppH triggers messenger RNA degradation by 5′ pyrophosphate removal. Nature; 451: 355-8.
Deana A, Belasco J G (2005) Lost in translation: the influence of ribosomes on bacterial mRNA decay. Genes Dev; 19: 2526-33.
Donovan W P, Kushner S R (1986) Polynucleotide phosphorylase and ribonuclease II are required for cell viability and mRNA turnover in Escherichia coli K-12. Proc Natl Acad Sci USA; 83: 120-4.
Dreyfus M, Regnier P (2002) The poly(A) tail of mRNAs: bodyguard in eukaryotes, scavenger in bacteria. Cell; 111: 611-3.
Freier S M, Kierzek R, Jaeger J A, Sugimoto N, Caruthers M H, Neilson T, Turner D H (1986) Improved free-energy parameters for predictions of RNA duplex stability. Proc Natl Acad Sci USA; 83: 9373-7.
Franch T, Gultyaev A P, Gerdes K (1997) Programmed cell death by hok/sok of plasmid R1: processing at the hok mRNA 3′-end triggers structural rearrangements that allow translation and antisense RNA binding. J Mol Biol; 273: 38-51.
Gerdes K, Thisted T, Martinussen J (1990) Mechanism of post-segregational killing by the hok/sok system of plasmid R1: sok antisense RNA regulates formation of a hok mRNA species correlated with killing of plasmid-free cells. Mol Microbiol; 4: 1807-18.
Guillier M, Gottesman S, Storz G (2006) Modulating the outer membrane with small RNAs. Genes Dev; 20: 2338-48.
Jacquet M, Kepes A (1971) Initiation, elongation and inactivation of lac messenger RNA in Escherichia coli studied studied by measurement of its beta-galactosidase synthesizing capacity in vivo. J Mol Biol; 60: 453-72.
Keiler K C, Waller P R, Sauer R T (1996) Role of a peptide tagging system in degradation of proteins synthesized from damaged messenger RNA. Science; 271: 990-3.
Komarova A V, Tchufistova L S, Dreyfus M, Boni I V (2005) AU-rich sequences within 5′ untranslated leaders enhance translation and stabilize mRNA in Escherichia coli. J Bacteriol; 187: 1344-9.
Kushner S R (2002) mRNA decay in Escherichia coli comes of age. J Bacteriol; 184: 4658-65.
Lemm I, Ross J (2002) Regulation of c-myc mRNA decay by translational pausing in a codon instability determinant. Mol Cell Biol; 22: 3959-69.
Mitarai N, Sneppen K, Pedersen S (2008) Ribosome collisions and translation efficiency: optimization by codon usage and mRNA destabilization. J Mol Biol; 382: 236-45.
Moore S D, Sauer R T (2005) Ribosome rescue: tmRNA tagging activity and capacity in Escherichia coli. Mol Microbiol; 58: 456-66.
Nilsson G, Belasco J G, Cohen S N, von Gabain A (1984) Growth-rate dependent regulation of mRNA stability in Escherichia coli. Nature; 312: 75-7.
Oppenheim A B, Rattray A J, Bubunenko M, Thomason L C, Court D L (2004) In vivo recombineering of bacteriophage lambda by PCR fragments and single-strand oligonucleotides. Virology; 319: 185-9.
Pedersen S, Reeh S, Friesen J D (1978) Functional mRNA half lives in E. coli. Mol Gen Genet; 166: 329-36.
Petersen C (1987) The functional stability of the lacZ transcript is sensitive towards sequence alterations immediately downstream of the ribosome-binding site. Mol Gen Genet; 209: 179-87.
Rasmussen A A, Eriksen M, Gilany K, Udesen C, Franch T, Petersen C, Valentin-Hansen P (2005). Regulation of ompA mRNA stability: the role of a small regulatory RNA in growth phase-dependent control. Mol Microbiol; 58: 1421-9.
Ringquist S, Shinedling S, Barrick D, Green L, Binkley J, Stormo G D, Gold L (1992). Translation initiation in Escherichia coli: sequences within the ribosome-binding site. Mol. Microbiol; 9: 1219-29.
Shultz J, Silhavy T J, Berman M L, Fiil N, Emr S D (1982) A previously unidentified gene in the spc operon of Escherichia coli K12 specifies a component of the protein export machinery. Cell; 31:227-35.
Sørensen M A, Kurland C G, Pedersen S (1989) Codon usage determines translation rate in Escherichia coli. J Mol Biol; 207: 365-77.
Sørensen M A, Pedersen S (1991) Absolute in vivo translation rates of individual codons in Escherichia coli. The two glutamic acid codons GAA and GAG are translated with a threefold difference in rate. J Mol Biol; 222: 265-80.
Vind J, Sørensen M A, Rasmussen M D, Pedersen S (1993) Synthesis of proteins in Escherichia coli is limited by the concentration of free ribosomes. Expression from reporter genes does not always reflect functional mRNA levels. J Mol Biol; 231: 678-88.
Yarchuk O, Jacques N, Guillerez J, Dreyfus M (1992) Interdependence of translation, transcription and mRNA degradation in the lacZ gene. J Mol Biol; 226: 581-96.
Sørensen M A, Pedersen S (1991) Absolute in vivo translation rates of individual codons in Escherichia coli. The two glutamic acid codons GAA and GAG are translated with a threefold difference in rate. J Mol Biol; 222: 265-80.
Vind J, Sørensen M A, Rasmussen M D, Pedersen S (1993) Synthesis of proteins in Escherichia coli is limited by the concentration of free ribosomes. Expression from reporter genes does not always reflect functional mRNA levels. J Mol Biol; 231: 678-88.
Oppenheim A B, Rattray A J, Bubunenko M, Thomason L C, Court D L (2004) In vivo recombineering of bacteriophage lambda by PCR fragments and single-strand oligonucleotides. Virology; 319: 185-9.
Sharan S K, Thomason L C, Kuznetsov S G, Court D L (2009) Recombineering: a homologous recombination-based method of genetic engineering. Nat Protoc; 4: 206-23.

1. A method to increase the production of a desired peptide in a cell by increasing the half-life of the mRNA transcript from the gene encoding the peptide, said method characterized in that one or more slowly translated codons are introduced in the gene 45-72 codons down-stream of the start site of the open reading frame, wherein the one or more slowly translated codons are selected so that the encoded amino acid sequence of the peptide is unchanged as compared to the wild type peptide.

2. Method according to claim 1, wherein the one or more slowly translated codons are introduced in the gene 45-66 codons down-stream of the start site of the open reading frame.

3. Method according to claim 1, wherein the cell is selected from the group consisting of an eukaryotic cell, selected from the group consisting of a vertebrate cell and a mammalian cell, and a microorganism selected from the group consisting of bacteria, fungi and algae.

4. Method according to claim 1, wherein the half-life is further increased by introducing one or more quickly translated codons in the gene 20 or less codons down-stream of the start site of the open reading frame.

5. Method according to claim 1, wherein the gene is lacZ gene.

6. A method of increasing the half-life of a mRNA transcript from a gene encoding a peptide, said method characterized in that one or more slowly translated codons are introduced in the gene 45-72 codons down-stream of the start site of the open reading frame, wherein the one or more slowly translated codons are selected so that the encoded amino acid sequence of the peptide is unchanged as compared to the wild type peptide.

7. Method according to claim 6, wherein the one or more slowly translated codons are introduced in the gene 45-66 codons down-stream of the start site of the open reading frame.

8. Method according to claim 6, wherein the one or more slowly translated codons are selected from codons that are translated with a rate of less than 6 codons per sec.

9. Method according to claim 6, wherein the one or more slowly translated codons are selected from codons that are translated with a rate of less than 4 codons per sec.

10. Method according to claim 6, wherein the one or more slowly translated codons are selected from codons that are translated with a rate of less than 3 codons per sec.

11. Method according to claim 6, wherein the half-life is further increased by introducing one or more quickly translated codons in the gene 20 or less codons down-stream of the start site of the open reading frame.

12. A method of decreasing the half-life of a mRNA transcript from a gene encoding a peptide, said method characterized in that one or more slowly translated codons are introduced in the gene 20 or less codons down-stream of the start site of the open reading frame, wherein the one or more slowly translated codons are selected so that the encoded amino acid sequence of the peptide is unchanged as compared to the wild type peptide.

13. Method according to claim 12, wherein the half-life is further decreased by introducing one or more quickly translated codons in the gene 45 or more codons down-stream of the start site of the open reading frame.

14. Method according to claim 12, wherein the half-life is further decreased by introducing one or more quickly translated codons in the gene 20 to 45 codons downstream of the start site of the open reading frame.

15. A method to decrease the production of a desired peptide in a cell by decreasing the half-life of the mRNA transcript from the gene encoding the peptide, said method characterized in that one or more slowly translated codons are introduced in the gene 20 or less codons down-stream of the start site of the open reading frame, wherein the one or more quickly translated codons are selected so that the encoded amino acid sequence of the peptide is unchanged as compared to the wild type peptide.

16. Method according to claim 15, wherein the one or more slowly translated codons are introduced in the gene 1-20, preferably 4-18, more preferably 5-15, and most preferably 6-15, codons down-stream of the start site of the open reading frame.

17. Method according to claim 15, wherein the one or more quickly translated codons are selected from codons that are translated with a rate of more than 6 codons per sec.

18. Method according to claim 15, wherein the half-life is further decreased by introducing one or more quickly translated codons in the gene 45 or more codons down-stream of the start site of the open reading frame.

19. Method according to claim 15, wherein the half-life is further decreased by introducing one or more quickly translated codons in the gene 20 to 45 codons down-stream of the start site of the open reading frame.

20. A recombinant vector for increasing the production of a desired peptide in a cell, said vector comprising a DNA sequence encoding the peptide, wherein the DNA sequence has an open reading frame with one or more slowly translated codons introduced 45-72 codons down-stream of the start site of the open reading frame, said one or more slowly translated codons being selected so that the encoded amino acid sequence of the peptide is unchanged as compared to the wild type peptide.

21. The vector of claim 20, wherein the one or more slowly translated codons are introduced in the gene 45-66 codons down-stream of the start site of the open reading frame.

22. The vector of claim 20, wherein one or more quickly translated codons are introduced 20 or less codons down-stream of the start site of the open reading frame.

23. A host cell transformed with a vector of claim 20.

24. A recombinant vector for decreasing the half-life of a mRNA transcribed from the vector encoding a peptide, said vector comprising a DNA sequence with an open reading frame having one or more slowly translated codons introduced 20 or less codons down-stream of the start site of the open reading frame, wherein the one or more slowly translated codons are selected so that the encoded amino acid sequence of the peptide is unchanged as compared to the wild type peptide.

25. A host cell transformed with a vector of claim 24.
